(19) United States
(12) Patent Application Publication
(10) Pub. No.: US 2023/0287039 A1
(43) Pub. Date: Sep. 14, 2023

(54) PROCESS FOR PRODUCING SINGLE CELL PROTEIN

(71) Applicant: Unibio A/S, Roskilde (DK)

(72) Inventors: Subir Kumar Nandy, Kongens Lyngby (DK); Ib Christensen, Allerød (DK)

(21) Appl. No.: 18/004,584

(22) PCT Filed: Jul. 6, 2021
(86) PCT No.: PCT/EP2021/068582

§ 371 Date: Jan. 6, 2023

(30) Foreign Application Priority Data
Jul. 7, 2020 (DK) PA 2020 00816

(51) Int. Cl. C07K 1/04; C12M 1/00

(52) U.S. Cl. CPC C07K 1/045; C12M 29/18

(57) ABSTRACT

The present invention relates to a process for providing a first reaction product by a first fermentation process conducted in a first Loop reactor, the method comprising the steps of: (i) adding an inoculum comprising one or more methanogenic microorganism to the first Loop reactor providing a first inoculated fermentation medium; (ii) adding a gaseous hydrogen (H₂) to the first inoculated fermentation medium; (iii) adding a first carbon source to the first inoculated fermentation medium; (iv) allowing the first fermentation medium to ferment, providing the first reaction product; and (v) isolating the first reaction product provided in step (iv).

### TECHNICAL FIELD OF THE INVENTION

The present invention relates to an improved process for producing single cell protein (SCP). In particular, the present invention relates to providing an improved process for producing a single cell protein that makes the location of the fermentation process (and the fermentation reactor) independent from the recovery of natural gas; that is independent on fluctuation in costs for fossil fuels; that has a reduced impact on the environment and/or the atmosphere; with increased simplicity; increased productivity; and/or increased efficiency.

### BACKGROUND OF THE INVENTION

Due to an increased world population, humans have an increasing demand for a protein rich diet, and animals (pets or farmed animals) and fish are feed more with protein rich diets in order to speed up growth and development and for the wellbeing of the animal.

However, with an increasing world population and an increasing demand for proteins in the animal farming and pet industry, there is strong evidence that agriculture will not be able to meet this demand and that there is a serious risk of food shortage.

Industrial agriculture is marked by a high-water footprint, high land use, biodiversity destruction, general environmental degradation and contributes to climate change by the emission of about a third of all greenhouse gases.

To meet the increasing demand for protein, and at least some of the mentioned disadvantages of industrial agriculture, the production of single cell protein (SCP) has shown to be a very interesting candidate.

Single cell protein (SCP) may be grown by fermentation of biomass through the growth of the microorganisms on hydrocarbon, nitrogen, and other substrates. SCP production represents options of fail-safe mass food-production which can produce food reliably worldwide and even under harsh climate conditions.

SCP product may be used directly in food or feed products, e.g. as a liquid product or as a spray dried product. The SCP or the biomass may alternatively be further processed, e.g. by hydrolysis and/or separation, to provide special fractions, remove impurities, or concentrating components, before use in a food or feed product.

The microorganisms traditionally used for producing SCP are methylotrophic microorganisms or methanotrophic microorganisms. These microorganisms digest methane provided in the form of natural gas (as carbon source gas), and in the presence of an oxygen compound and a nitrogen compound and convert this to biomass that ends up as the SCP product.

Methane is the major component of natural gas and accounts for about 87% by volume. The major source of methane is the extraction of geological deposits. It is associated with other hydrocarbon fuels. In general, the sediments that generate natural gas are buried deeper and at higher temperatures than those that contain oil, which may make it more difficult to recover. Methane is generally transported in bulk by pipeline in its natural gas form, or LNG carriers in its liquefied form, or a few countries transport methane by truck.

Hence, some of the challenges of the presently provided processes for providing SCP are that the location of the process may be limited to the areas where the methane or the natural gas, is available. Alternatively, the methane or the natural gas should be transported to the SCP fermenter, which adds additional costs to the production. Furthermore, as mentioned above, the methane used is traditionally obtained from fossil fuels which may be a limiting factor, and subject to large fluctuations in costs and harmful effects on the environment and the atmosphere.

Moreover, fermentation processes based on the digestion of natural gas involve a co-fermentation of different types of microorganisms, since natural gas comprises minor amounts of different hydrocarbons other than methane that needs to be digested in order not to accumulate in the fermentation medium and causing the fermentation process to decrease in effectivity or perhaps even stop the fermentation process which subsequently may be restarted.

Hence, there is a need in the industry for an improved process of producing a single cell protein, and an improved process that solves the problems with the prior art would be advantageous.

In particular, there is a need for an improved process that will be independent of the location of extraction of natural gas, and hence, the presence of cheap methane gas, which process is not affected by fluctuating prices of fossil fuels; more environmental and/or atmosphere friendly, and which process is simpler, more efficient and/or more reliable, would be advantageous.

### SUMMARY OF THE INVENTION

Thus, an object of the present invention relates to an improved process of producing a single cell protein, and an improved process that solves the problems with the prior art would be advantageous.

In particular, it is an object of the present invention to provide an improved process of producing a single cell protein that solves the above-mentioned problems of the prior art with the location of the fermentation process (and the fermentation reactor); fluctuation in costs for fossil fuels; environmental and/or atmospheric challenges, simplicity, productivity, and efficiency.

Thus, one aspect of the invention relates to a process for providing a first reaction product by a first fermentation process conducted in a first Loop reactor, the method comprising the steps of:
(i) adding an inoculum comprising one or more methanogenic microorganism to the first Loop reactor providing a first inoculated fermentation medium;
(ii) adding gaseous hydrogen (H2) to the first inoculated fermentation medium;
(iii) adding a first gaseous carbon source, such as a gaseous carbon monoxide (CO); a gaseous carbon dioxide (CO2) or a combination hereof, to the first inoculated fermentation medium;
(iv) allowing the first fermentation medium to ferment, providing the first reaction product; and
(v) isolating the first reaction product provided in step (iv).

Another aspect of the present invention relates to a process for producing a second single cell protein comprising the steps of:
(a) providing a gaseous hydrogen gas (H2);
(b) mixing the hydrogen gas from step (a) with a first gaseous carbon source, such as carbon monoxide (CO); carbon dioxide (CO2) or a combination hereof providing a C1-compound;
(c) adding the C1-compound provided in step (b) to a second Loop reactor comprising one or more microorganisms capable of metabolizing the C1-compound providing a second inoculated fermentation medium;
(d) allowing the second inoculated fermentation medium to ferment, in a second fermentation process, and converting the C1-compound into a biomass material, and
(e) isolating the biomass material provided in step (c) and providing the second single cell protein.

Yet another aspect of the present invention relates to a Loop reactor comprising a loop-part and a top tank, said loop-part comprising a downflow part, connected to an upflow part via a U-part, wherein the loop-part comprises at least one inlet for injecting a gaseous hydrogen (H₂)

Still another aspect of the present invention relates to a single cell protein composition comprising a first single cell protein according to the present invention, and a second single cell protein according to the present invention.

Furthermore, an aspect of the present invention relates to the use of the single cell protein composition according to the present invention, as an ingredient in a feed product for an animal.

The present invention will now be described in more detail in the following.

### DETAILED DESCRIPTION OF THE INVENTION

The inventors of the present invention found that the presently available processes for providing single cell protein (SCP) had several undesirable restrictions, undesirable drawbacks, and challenges that have a negative effect on the usage of the technology and the productibility of the process of producing single cell protein (SCP). Hence, the inventors of the present invention surprisingly found a process for disconnecting the process from a location having available carbon source (e.g. methane), which also shows to be more environmental and/or atmosphere friendly, and which process is simpler, and/or more efficient.

A preferred embodiment of the present invention relates to the process for providing a first reaction product by a first fermentation process conducted in a first Loop reactor, the method comprising the steps of:
(i) adding an inoculum comprising one or more methanogenic microorganism to the first Loop reactor providing a first inoculated fermentation medium;
(ii) adding gaseous hydrogen (H2) to the first inoculated fermentation medium;
(iii) adding a first gaseous carbon source, such as a gaseous carbon monoxide (CO); a gaseous carbon dioxide (CO2) or a combination hereof, to the first inoculated fermentation medium;
(iv) allowing the first fermentation medium to ferment, providing the first reaction product; and
(v) isolating the first reaction product provided in step (iv).

In the context of the present invention, the term “loop” relates to a loop reactor comprising a loop-part and a top tank (gas/liquid separation tank). The top tank may comprise a vent tube for discharging effluent gasses from the top tank. The loop-part may comprise a substantially vertical downflow part connected to a substantial vertical upflow part via a horizontal part or a U-part In a preferred embodiment of the present invention, the loop-part comprises a circulation pump for circulating the fermentation medium, when present in the fermenter.

In yet an embodiment of the present invention the loop-part having a length which may be longer, preferably substantially longer, than the length and/or the height of the top tank.

In a further embodiment of the present invention, the top tank comprises a volume that is larger than the volume of the loop-part. Preferably, the fermentation reactor comprises a loop-part having a length which may be longer, preferably substantially longer, than the length and/or the height of the top tank, and the top tank comprises a volume which is larger than the volume of the loop-part.

In an embodiment of the present invention, the loop-part of the present invention may relate to at least one downflow part, at least one upflow part as well as at least one connecting part.

In the present context, the term “U-part” relates to bend provided in the bottom part of the fermentation reactor or the loop reactor connecting the lower ends of the upflow part and the downflow part.

Preferably, the one or more upflow part(s) and the one or more downflow part(s) are vertical or substantially vertical.

The loop reactor according to the present invention may be designed as a vertical loop reactor or a horizontal loop reactor.

In an embodiment of the present invention, the fermentation reactor may be a vertical loop reactor. A vertical loop reactor may relate to a loop reactor having a main part of the U-part in vertical, or substantially vertical, position, relative to the horizontal position. In an embodiment of the present invention, the fermentation reactor comprises a main part of the U-part in vertical, or substantially vertical, position.

In another embodiment of the present invention, the fermentation reactor may be a horizontal loop reactor. A horizontal loop reactor may relate to a loop reactor having a main part of the U-part in horizontal, or substantially horizontal, position relative to the vertical position. In an embodiment of the present invention, the fermentation reactor comprises a main part of the U-part in horizontal, or substantially horizontal, position.

Preferably, the fermentation reactor may be designed as a vertical loop reactor.

In the context of the present invention the term “main part” relates to at least 51% (v/v) of the U-part having the desired position; such as at least 55% (v/v); e.g. at least 60% (v/v); such as at least 65% (v/v); e.g. at least 70% (v/v); such as at least 75% (v/v); e.g. at least 80% (v/v); such as at least 85% (v/v); e.g. at least 90% (v/v); such as at least 95% (v/v); e.g. at least 98% (v/v).

In the present context, the term “top tank” relates to a container located at the top of the fermentation reactor and responsible for removal of effluent gas from the fermentation liquid. Preferably, the top tank is during operation/fermentation only partly filled with fermentation liquid. In an embodiment of the present invention, the term “partly filled with fermentation liquid” relates to a 90:10 ratio between fermentation liquid and gas; such as an 80:20 ratio; e.g. a 70:30 ratio; such as a 60:40 ratio; e.g. a 50:50; such as a 40:60 ratio; e.g. a 30:70 ratio; such as a 20:80 ratio; e.g. a 10:90 ratio.

In the context of the present invention, the “visual inspection means” relates to one or more means allowing the skilled person to obtain direct information, e.g. on flowability and/or on the foaming characteristics, in the top tank and/or in the loop-part.

In an embodiment of the present invention, the direct information may be real-time information on the foaming characteristics in the top tank.

In a further embodiment of the present invention, the first carbon source may be a first gaseous carbon source; or a first liquid carbon source. Preferably, the first carbon source is a first gaseous carbon source.

The first gaseous carbon source may be a gaseous carbon monoxide (CO); a gaseous carbon dioxide (CO₂); or a combination hereof.

The addition of or the flow of:
the gaseous hydrogen (H₂) and/or
the first gaseous carbon source, e.g. the gaseous carbon monooxide (CO); the gaseous carbon dioxide (CO₂); or the combination hereof,
   to the first inoculated fermentation medium present in the first loop reactor may be controlled by the need of hydrogen (H₂) necessary for optimized production and/or the hydrogen (H₂) consumption of the one or more methanogenic microorganism.

Cultivation and fermentation of methanogenic microorganisms are generally known to the skilled person.

In an embodiment of the present invention the gaseous hydrogen (H₂) and/or the first gaseous carbon source, such as gaseous carbon monoxide (CO); gaseous carbon dioxide (CO₂); or a combination hereof, may be continuously added to the first inoculated fermentation medium during the fermentation process.

In the context of the present invention, the term “hydrogen” relates to the chemical compound dihydrogen (H₂). The hydrogen (H₂) may be provided in a gaseous form.

In an embodiment of the present invention the gaseous hydrogen may be provided from the electrolysis of water; obtained from natural sources, like earth reserves; microbially produced; or chemically produced.

The electrolysis of water results in the decomposition of water molecules into oxygen and hydrogen gas due to the passage of an electric current. A DC-electrical power source connected to two electrodes, or two plates (typically made from some inert metal such as platinum or iridium) may be placed in the water and the hydrogen gas may easily be collected from the cathode.

In an embodiment of the present invention the first gaseous carbon source, such as the gaseous carbon monoxide (CO) and/or the gaseous carbon dioxide (CO₂) may be obtained from a carbon capture process, or a chemical process, an enzymatic process or microbial process.

The methanogenic microorganism may be a methanogenic archaeon, a methanogenic bacterium, a methanogenic yeast, a methanogenic fungus, or a combination hereof.

In an embodiment of the present invention, the methanogenic microorganism may be a prokaryotic organism. Preferably, the methanogenic microorganism may be a methanogenic archaeon.

The methanogenic archaeon may preferably be selected from the group consisting of Methanobacterium bryantii; Methanobacterium formicicum; Methanobacterium thermoalcaliphium; Methanothermobacter wolfeii; Methanobrevibacter smithii; Methanobrevibacter ruminantium; Methanococcus voltae; Methanomicrobium mobile; Methanolacinia paynteri; Methanospirillum hungatei; Methanosarcina acetivorans; Methanosarcina barkeri; Methanosarcina mazei; Methanosarcina thermophile; Methanococcoides methylutens; Methanosaeta concilii (soehngenii); and Methanosaeta thermophila.

In an embodiment of the present invention, the fermentation process may be a batch fermentation, a fed-batch fermentation, or a continuous fermentation. Preferably the fermentation process may be continuous.

For commercial production of e.g. SCP the fermentation process may involve 3 fermentation stages:
A batch fermentation; which is the initial propagation of the organisms where all materials except the organisms, required are decontaminated by autoclaving before, loaded to the reactor together with the organisms and the process starts. The organism used goes through all the growth phases (lag phase, log or exponential phase, and steady state phase. Under this operation mode, conditions are continuously changed with time under an unsteady-state system and require a lot of work and involvement.
A fed-batch fermentation; is a biotechnological operational process where one or more nutrients are feed to the bioreactor during cultivation and in which the product(s) remain in the bioreactor until the end of the run. The fed-batch fermentation may traditionally follow the batch fermentation and may be provided to achieve very high cell concentrations of the organism before turning the process into a continuous fermentation since batch fermentation would require inhibitory high concentrations of nutrients and would therefore, be very difficult or not even possible. The fed-batch fermentation may be used for preparing the cell culture for continuous fermentation.
A continuous fermentation; is the production model of the fermentation process where feeding the microorganism with sterile fermentation medium which is used for the cultivation of the organism, and at the same time removing part of the fermentation medium including the biomass from the system. This makes a unique feature of a continuous supply of biomass that may be used as a single cell protein or fractionated to various fractions.

The production mode of the process according to the present invention may preferably be run as a continuous fermentation process. Preferably, the continuous fermentation process follows a batch fermentation and/or a fed-batch fermentation process, starting with adding water, necessary nutrient salts, and the microorganisms to the fermentation reactor creating a first inoculated fermentation medium, and the batch and/or fed-batch fermentation process may be started. When a sufficient biomass content has been reached, the continuous fermentation process may be started

For financial reasons, there may be an interest and a drive in the industry to start the continuous and steady state fermentation as quickly as possible to save time and costs and provide the SCP product faster and profitable to the market but also enable one to determine the relations between the environmental conditions and microbial behavior including both genetic and phenotypic expression.

The first inoculated fermentation medium may be allowed to ferment during batch fermentation and/or fed-batch fermentation for a period in the range of 6 hours to 6 days; such as for a period of 12 hours to 5 days; e.g. for a period of 1-4 days, such as for a period of 2-3 days.

The first inoculated fermentation medium may be circulated in the first fermentation reactor, preferably by a first pressure controlling device, and the addition of substrates like gaseous hydrogen (H₂) and carbon source may be initiated, and the first fermentation process may be started. When the density of microorganisms has reached a concentration of approximately 0.5-10%, and preferably 1-5% (by dry weight) the first fermentation process may be shifted to a continuous fermentation process where the first inoculated fermentation medium may continuously be withdrawn from the first fermentation reactor, e.g. from the top tank and/or from the U-part and subjected to downstream processing providing the desired first reaction products. Simultaneously, with continuously withdrawing the first inoculated fermentation medium from the first fermentation reactor a substrate comprising water, salts and nutrients may be added.

In an embodiment of the present invention, the first inoculated fermentation medium may during continuous fermentation be allowed to ferment for a period of at least 3 days, such as for at least 6 days, e.g. for at least 2 weeks, such as for at least 4 weeks, e.g. for at least 1½ months, such as for at least 2 months, e.g. for at least 3 months.

The first inoculated fermentation medium may during continuous fermentation ferment until the cultivation is stopped forcefully or manually due to the need for maintenance; microbial contamination; chemical contamination; problems with substrates or the like.

In an embodiment of the present invention, the first inoculated fermentation medium may be allowed to ferment at a temperature in the range of 25-60° C.; such as in the range of 30-50° C.; e.g. in the range of 35-45° C.; such as in the range of 40-43° C.

The first fermentation process relates to the fermentation of a methanogenic microorganism and provides a first reaction product.

In the contest of the present invention the term “first reaction product” relates to one or more product(s) obtained from the first fermentation process by the action of a methanogenic microorganism.

In an embodiment of the present invention, the first reaction product provided in step (v) may be a first biomass material; a first single cell protein; a C1-compound; or a combination hereof.

In yet an embodiment of the present invention, the first reaction product comprises a single cell protein.

The first biomass material and/or the first single cell protein may comprise one or more methanogenic microorganism(s).

In an embodiment of the present invention, the C1 compound may be methane, methanol, or derivates thereof. Preferably, the C1 compound may be methane.

Preferably, several first reaction products may be obtained from the first fermentation process.

In an embodiment of the present invention, the first reaction product provided in step (v) may comprise a combination of a first single cell protein and a C1-compound.

The first reaction product may comprise a C1 compound and the C1 compound may be added to a second loop reactor, the second loop reactor comprising a second inoculated fermentation medium, the second inoculated fermentation medium comprising one or more microorganisms capable of metabolizing the C1 compound and converting the C1 compound into a second reaction product by a second fermentation process.

In the contest of the present invention the term “second reaction product” relates to one or more product(s) obtained from the second fermentation process by the action of one or more microorganisms capable of metabolizing the C1 compound.

The second reaction product may be a second single cell protein, a second biomass material, CO₂, or a combination hereof.

The second reaction product may be a second single cell protein, a second biomass material, or a fraction hereof.

In an embodiment of the present invention, the second reaction product may be a combination of C02, a single cell protein, or a fraction of a single cell protein.

A fraction of a single cell protein or a fraction of a biomass product may be obtained by a method described in WO 2018/115042 as well as downstream processing of first and/or second reaction products that may be performed according to the process described in WO 2018/115042.

The one or more microorganisms capable of metabolizing the C1 compound may be one or more aerobic microorganism.

In an embodiment of the present invention, the one or more aerobic microorganisms may be one or more aerobic methanotrophic microorganisms and/or one or more aerobic methylotrophic microorganism. Preferably, one or more aerobic methanotrophic microorganisms or one or more aerobic methylotrophic microorganism may be one or more aerobic methanotrophic bacteria and/or one or more aerobic methylotrophic bacteria, respectively.

In a further embodiment of the present invention, the one or more microorganisms capable of metabolizing the C1 compound may not be a recombinant microorganism.

In the context of the present invention, the term “recombinant microorganism” relates to a genetically modified organism (GMO) whose genetic material has been altered using genetic engineering techniques. The recombinant microorganism may be considered in contrast to genetic alterations that occur naturally in the microorganism, e.g. by mating and/or natural recombination.

Preferably, the one or more microorganism capable of metabolizing the C1 compound may be one or more naturally occurring microorganism.

In an embodiment of the present invention the one or more microorganism capable of metabolizing the C1 compound may be a bacteria, such as a methanotrophic or a methylotropic bacteria; a yeast, such as a methanotrophic or a methylotropic yeast; a fungus, such as a methanotrophic or a methylotropic fungus; or a combination hereof.

In the context of the present invention the term “naturally occurring microorganism” relates to a microorganism whose genetic material has not been altered using genetic engineering techniques. Natural modifications or alterations in the genetic material of a microorganism may be covered by the term “naturally occurring microorganism”.

In an embodiment of the present invention, the one or more aerobic methanotrophic bacteria may be a Methylococcus. Preferably, the Methylococcus is M. capsulatus, more preferably, the M. capsulatus may be M. capsulatus (Bath); even more preferably the M. capsulatus (Bath) identified under NCIMB 11132.

In a further embodiment of the present invention, the one or more microorganisms capable of metabolizing the C1 compound may be provided in combination with another microorganism (as in co-fermentation).

The other microorganism in the co-fermentation may be selected according to possible impurities, such as carbon compounds other than C1, that are not metabolized or digested by the one or more microorganisms capable of metabolizing the C1 according to the present invention, and thus may accumulate in the second inoculated fermentation medium during the second fermentation process.

In an embodiment of the present invention the co-fermentation may be provided as a combination of the one or more microorganisms capable of metabolizing the C1, preferably, M. capsulatus, in combination with one or more microorganism selected from Ralstonia sp.; Bacillus brevis; Brevibacillus agri; Alcaligenes acidovorans; Aneurinibacillus danicus and Bacillus firmus.

In particular, co-fermentation according to the present invention may relate to a co-fermentation comprising the combination of M. capsulatus (preferably, NCIMB 11132); A. acidovorans (preferably NCIMB 13287); B. firmus (preferably NCIMB 13289); and A. danicus (preferably NCIMB 13288).]

In an embodiment of the present invention the yeast may be a methanotrophic or a methylotropic yeast. Preferably, the yeast may be selected from Pichia pastoris; Komagataella phaffii; Komagataella pastoris; and/or Komagataella pseudopastoris.

In an embodiment of the present invention, the second biomass material and/or the second single cell protein may comprise one or more methanotrophic microorganisms and/or one or more methylotrophic microorganisms.

In a further embodiment of the present invention, the first single cell protein and the second single cell protein may be mixed providing a combined single cell protein.

In an embodiment of the present invention the second inoculated fermentation medium may be allowed to ferment during batch fermentation for a period in the range of 6 hours to 6 days; such as for a period of 12 hours to 5 days; e.g. for a period of 1-4 days, such as for a period of 2-3 days.

During production mode, the second fermentation process according to the present invention may preferably be run as a continuous fermentation process. Preferably, the continuous fermentation process of the second inoculated fermentation medium follows a batch fermentation and/or a fed-batch fermentation process, starting by adding water, necessary nutrient salts and the microorganisms (including one or more microorganisms capable of metabolizing the C1) to the second fermentation reactor creating the second inoculated fermentation medium, and the batch and/or fed-batch fermentation process may be started.

The second inoculated fermentation medium may be circulated in the fermentation reactor, preferably by a first pressure controlling device, and the addition of substrates, like a gaseous C1 compound, may be initiated, and fermentation may be started. When the density of microorganisms in the second fermentation reactor has reached a concentration of approximately 0.5-10%, and preferably 1-5% (by dry weight) the second fermentation process may be shifted to a continuous fermentation process where the second inoculated fermentation medium may continuously be withdrawn from the second fermentation reactor, e.g. from the top tank and/or from the U-part and subjected to downstream processing providing the desired second reaction products. Simultaneously, with continuously withdrawing the second inoculated fermentation medium from the fermentation reactor, a substrate comprising water, salts and nutrients may be added.

In an embodiment of the present invention the second inoculated fermentation medium may during continuous fermentation be allowed to ferment for a period of at least 3 days, such as for at least 6 days, e.g. for at least 2 weeks, such as for at least 4 weeks, e.g. for at least 1½ months, such as for at least 2 months, e.g. for at least 3 months.

The second inoculated fermentation medium may during continuous fermentation ferment until the cultivation is stopped forcefully or manually due to the need for maintenance; microbial contamination; chemical contamination; problems with substrates or the like. In an embodiment of the present invention, the second inoculated fermentation medium may be allowed to ferment at a temperature in the range of 25-60° C.; such as in the range of 30-50° C.; e.g. in the range of 35-45° C.; such as in the range of 40-43° C.

In yet an embodiment of the present invention the second fermentation process may comprise addition of carbon dioxide (CO₂) to the second inoculated fermentation medium.

In an embodiment of the present invention, one or more methanotrophic microorganism and/or one or more methylotrophic microorganism according to the present invention may be added to the first inoculated fermentation medium providing a co-fermentation between the one or more methanogenic microorganism; and the one or more methanotrophic microorganism and/or one or more methylotrophic microorganism. One or more methanotrophic microorganism and/or one or more methylotrophic microorganism may then concerting the C1 compound generated from the first fermentation process directly from the first inoculated fermentation medium before the isolation step (v).

In yet an embodiment of the present invention, gaseous oxygen (O₂) may be added to the second inoculated fermentation medium.

As mentioned earlier in respect of the first fermentation process hydrogen (H2) is added to the first fermentation reactor and the hydrogen (H2) may be provided from the electrolysis of water which is decomposed into oxygen (O₂) gas and hydrogen (H₂) gas due to the passage of an electric current.

In an embodiment of the present invention, the gaseous oxygen (O₂) is provided from hydrolyzing water resulting in gaseous hydrogen (H₂), which gaseous hydrogen (H₂) may be added to the first inoculated fermentation medium and the gaseous oxygen (O₂) may be added to the second inoculated fermentation medium.

When the gaseous hydrogen is provided from the electrolysis of water, oxygen is obtained too. The oxygen obtained may be used in the second fermentation process for providing a second reaction product, e.g. a second single cell protein comprising a methanotrophic microorganism or a methylotrophic microorganism.

In an embodiment of the present invention, the CO₂ produced in the second fermentation process may be recycled to the first inoculated fermentation medium and/or to the second inoculated fermentation medium.

A preferred embodiment of the present invention relates to a process for producing a second single cell protein comprising the steps of:
(a) providing a gaseous hydrogen gas (H₂);
(b) mixing the hydrogen gas from step (a) with a first carbon source, e.g. a first gaseous carbon source, such as carbon monoxide (CO); carbon dioxide (CO₂) or a combination hereof, providing a C1 compound;
(c) adding the C1 compound provided in step (b) to a second loop reactor comprising one or more microorganisms capable of metabolizing the C1-compound providing a second inoculated fermentation medium;
(d) allowing the second inoculated fermentation medium to ferment, in a second fermentation process, and converting the C1 compound into a second biomass material; and
(e) isolating the second biomass material provided in step (c) and providing the second single cell protein.

In an embodiment of the present invention, the C1 compound provided in step (b) may be obtained according to the first fermentation process described above.

In yet an embodiment of the present invention, the gaseous hydrogen gas (H₂) provided in step (a) may be obtained by subjecting the water to a water decomposition treatment resulting in splitting water molecules (H₂O) into hydrogen gas (H₂) fraction and an oxygen gas (O₂) fraction.

Preferably, the water decomposition treatment may be electrolysis.

Electrolysis is a process where an electrical power source is connected to two electrodes or two plates (typically made from some inert metal, such as platinum or iridium) which are placed in the water. When the electrical power source is activated hydrogen (H₂) will appear at the cathode (where electrons enter the water), and oxygen will appear at the anode. Assuming ideal faradaic efficiency, the amount of hydrogen generated is twice the amount of oxygen, and both are proportional to the total electrical charge conducted by the solution.

During the electrolysis of water, oxygen is will appear at the anode and may be isolated and added to the second inoculated fermentation medium.

Carbon dioxide (CO₂) may be generated from the second fermentation process may be recirculated.

A preferred embodiment of the present invention relates to a loop reactor comprising a loop-part and a top tank, said loop-part comprising a downflow part, connected to an upflow part via a horizontal part, a substantial horizontal part, or a U-part, wherein the loop-part comprises at least one inlet for injecting gaseous hydrogen (H₂) In an embodiment of the present invention the loop part may further comprise at least one inlet for injecting a gaseous carbon monoxide (CO); a gaseous carbon dioxide (CO2) or a combination hereof.

Preferably, the loop reactor comprises a circulation pump.

In an embodiment of the present invention, a first pressure controlling device may be provided in the loop part of the loop reactor. Preferably the circulation pump may act as a first pressure controlling device.

The first pressure controlling device may be provided in the upper part of the downflow part of the loop part of the loop reactor.

Downstream from the first pressure controlling device a second pressure controlling device may be provided. Preferably, the second pressure controlling device is provided in the upper part of the upflow part.

The second pressure controlling device may be selected from the group consisting of a narrowing of the diameter/cross section of a section of the upper part of the upflow part; a plate with holes; jets; nozzles; a valve; a hydro cyclone; or a pump (such as a propeller pump, a lobe pump or a turbine pump).

The first pressure controlling device may pump a fermentation medium towards the second pressure controlling device which generates an increased pressure on the fermentation medium between the first pressure controlling device and the second pressure controlling device. This increased pressure may increase the mass transfer of gas from the undissolved state to dissolved state and become available for microbial consumption

In an embodiment of the present invention the loop reactor may comprise at least one inactive mixer and/or at least one active mixer.

The top tank of the loop reactor may comprise:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop-part into the top tank; and

The top tank may further comprise a vent tube for discharging effluent gasses from the top tank.

In an embodiment of the present invention, the top tank further comprises a visual inspection means.

In a further embodiment of the present invention, the loop-part comprises a visual inspection means.

The visual inspection means may be provided in the loop part in order to control the flow of the fermentation medium and/or turbulence of the fermentation medium in the lop part to ensure an optimized fermentation and an improved productivity of the fermentation process.

The visual inspection means may be provided in the top tank in order to control foaming and/or turbulence of the fermentation liquid in the top tank to ensure an optimized degassing of effluent gasses and hence, an improved productivity of the fermentation process.

Preferably, the visual inspection means may be placed with a horizontal or substantial horizontal inspection view into the top tank.

The visual inspection means may be placed on the side of the top tank allowing a combined view above the surface of a fermentation liquid and below the surface of the fermentation liquid.

Preferably, the visual inspection means may be placed in the end of the top tank.

Preferably, the visual inspection means may be placed at the end of the top tank providing a view from the first inlet (or the upflow part) towards the first outlet (or the downflow part).

In an embodiment of the present invention, the visual inspection means according to the present invention may be an inspection hole, the camera, or a combination of an inspection hole and a camera, such as an inline camera.

In yet an embodiment of the present invention, the inspection hole may be a sight glass.

The loop reactor may comprise at least one hydrogen (H₂) sensor. The Hydrogen sensor may provide information on the amount of dissolved and/or undissolved hydrogen (H₂) in the first inoculated fermentation medium. In this way, it may be possible to optimize the first fermentation process according to the present invention.

Further details of suitable modifications to the loop reactor and feature on how to run such loop reactor, and processing of resulting biomass may be as described in WO 2010/069313; WO 2000/70014; WO 2003/016460; WO 2018/158319; WO 2018/158322; WO 2018/115042 and WO 2017/080987 which are all incorporated by reference.

A preferred embodiment of the present invention relates to a combined single cell protein composition comprising a first single cell protein according to the present invention, and a second single cell protein according to the present invention.

Preferably, the first single cell protein comprises one or more methanogenic microorganisms.

Preferably, the second single cell protein comprises one or more a methanotrophic microorganism or a methylotrophic microorganism.

In an embodiment of the present invention, the combined single cell protein comprises a combination of
one or more methanogenic microorganism; and
one or more methanotrophic microorganisms or one or more methylotrophic microorganisms.

A preferred embodiment of the present invention relates to the use of the combined single cell protein composition according to the present invention, as an ingredient in a feed product for an animal or in a food product for a human.

The feed product may be a ruminant feed product, a fish feed product, a pig feed product, or a poultry feed product.

It should be noted that embodiments and features described in the context of one of the aspects of the present invention also apply to the other aspects of the invention.

All patent and non-patent references cited in the present application are hereby incorporated by reference in their entirety.

### REFERENCES

WO 2010/069313
WO 2000/70014
WO 2003/016460
WO 2018/158319
WO 2018/158322
WO 2018/115042
WO 2017/080987
WO 2018/132379

1. A process for providing a final reaction product by a first and second fermentation process conducted in a first and a second Loop reactor, the method comprising the steps of:
(i) adding an inoculum comprising one or more methanogenic microorganism to the first Loop reactor providing a first inoculated fermentation medium;
(ii) adding a gaseous hydrogen (H₂) to the first inoculated fermentation medium;
(iii) adding a first carbon source to the first inoculated fermentation medium;
(iv) allowing the first fermentation medium to ferment, providing a first reaction product; and
(v) isolating the first reaction product provided in step (iv)
wherein the first reaction product comprises a C1 compound and wherein the C1 compound is added to the second Loop reactor, the second Loop reactor comprising a second inoculated fermentation medium, the second inoculated fermentation medium comprising one or more microorganisms capable of metabolising the C1 compound and converting the C1 compound into the final reaction product by a second fermentation process wherein the final reaction product is a single cell protein.

2. The process according to claim 1, wherein the first carbon source is a first gaseous carbon source and, wherein the first gaseous carbon source is a gaseous carbon monoxide (CO); a gaseous carbon dioxide (CO₂); or a combination hereof.

3. The process according to claim 2, wherein the gaseous carbon source is obtained from a carbon capture process, a chemical process, an enzymatic process, or a microbial process.

4. The process according to anyone of the preceding claims, wherein the C1 compound is methane or methanol.

5. The process according to any one of the preceding claims, wherein the H₂ is provided from electrolysis or water, obtained from natural sources, or produced microbially or chemically.

6. The process according to claim 2, wherein the H₂ is provided from electrolysis of water, and wherein the O₂ thus obtained is added to the second inoculated fermentation medium or used in the second fermentation process.

7. The process according to any one of the preceding claims, wherein CO₂ generated from the second fermentation process is recirculated.

8. The process according to claim 8, wherein the CO₂ is recycled to the first inoculated fermentation medium and/or to the second inoculated fermentation medium.

9. A process for producing a single cell protein comprising the steps of:
(a) providing a gaseous hydrogen gas (H₂);
(b) mixing the hydrogen gas from step (a) with a carbon source, e.g. a gaseous carbon source, such as carbon monoxide (CO); carbon dioxide (CO₂) or a combination hereof, providing a C1 compound;
(c) adding the C1 compound provided in step (b) to a loop reactor comprising one or more microorganisms capable of metabolizing the C1-compound providing an inoculated fermentation medium;
(d) allowing the inoculated fermentation medium to ferment in a fermentation process, and converting the C1 compound into a biomass material; and
(e) isolating the biomass material provided in step (c) and providing the single cell protein.

10. The process according to claim 9, wherein the H₂ is provided from electrolysis of water, obtained from natural sources, or produced microbially or chemically.

11. The process according to claim 10, wherein the H₂ is provided from electrolysis of water, and wherein the O₂ thus obtained is added to the inoculated fermentation medium or used in the fermentation process.

12. The process according to any one of claims 9-11, wherein the gaseous carbon source is obtained from a carbon capture process, a chemical process, an enzymatic process, or a microbial process.

13. The process according to any one of claims 9-13, wherein CO₂ generated the fermentation process is recirculated.
